(19) United States
(12) Patent Application Publication
(10) Pub. No.: US 2012/0053172 A1
(43) Pub. Date: Mar. 1, 2012

(54) USE OF A COMBINATION OF DIAZOXIDE AND METFORMIN FOR TREATING OBESITY OR OBESITY RELATED DISORDERS

(75) Inventor: Johannes Marthijs Maria De Boer, Velp (NL)

(73) Assignee: Cooperatieve MIRzorg U.A.

(21) Appl. No.: 13/201,208

(22) PCT Filed: Feb. 12, 2010
(86) PCT No.: PCT/NL2010/050066

§ 371 Date: Nov. 11, 2011

Related U.S. Application Data
(60) Provisional application No. 61/151,947, filed on Feb. 12, 2009.

(51) Int. Cl. A61K 31/549; A61P 3/04; A61P 3/10

(52) U.S. Cl. 514/223.5

(57) ABSTRACT

The present invention relates to a method for suppressing the fasting plasma insulin level and/or postabsorptive insulin level in a mammal in need thereof, said method comprising administering to said mammal a pharmaceutically effective amount of a combination of a potassium channel activator and an anti-diabetic drug. The present invention also relates to a method for treating or preventing obesity, obesity related disorders and conditions and other disorders and conditions related to weight gain in a mammal in need thereof, said method comprising administering to said mammal a pharmaceutically effective amount of a combination of a potassium channel activator and an anti-diabetic drug.

### FIELD OF THE INVENTION

The present invention relates to a method for suppressing the fasting plasma and/or postabsorptive insulin levels in a mammal, in particular in a male mammal. The present invention also relates to a method for preventing or treating enhanced fasting and/or postabsorptive plasma insulin levels in a mammal, in particular in a male mammal. The present invention also relates to a method for preventing or treating obesity and obesity related disorders.

### BACKGROUND OF THE INVENTION

Obesity, which can be defined as a body weight more than 20% above the ideal body weight or even better by a Body Mass Index (BMI; expressed as the ratio of the mammal's weight and the square of its length) of 30 kg/m² or higher (cf. World Health Organization. Technical report series 894: “Obesity: preventing and managing the global epidemic.”, Geneva, World Health Organization, 2000, incorporated by reference), is a rapidly increasing global problem that urgently needs to be controlled. Obesity causes or exacerbates many health problems, both independently and in association with other disorders. The medical problems associated with obesity, which can be serious and life-threatening, include hypertension, type 2 diabetes mellitus, elevated plasma insulin concentrations; insulin resistance, dyslipidemias, hyperlipidemia, endometrial, breast-, prostate- and colon-cancer, osteoarthritis, respiratory complications, cholelithiasis, gallstones, arteriosclerosis, heart disorder, abnormal heart rhythms, and heart arrythmias. Reference is for example made to US 2006/276549 and WO 2006/124506 to Abbot Laboratories, both incorporated by reference.

Nowadays, three medical compounds are used for the treatment of obesity, i.e. Rimonabant (Acomplia®), Sibutramine (Meridia®) and Orlistat (Xenical®). Rimonabant is originally disclosed in U.S. Pat. No. 5,624,941 to Sanofi, incorporated by reference. Rimonabant is manufactured by Sanofi-Aventis and is a CB1 cannabinoid receptor antagonist. However, the European Medicines Agency (EMEA) recommended in October 2008 to temporarily suspend the marketing authorisation of Rimonanbant. In November 2008, Sanofi announced that clinical studies with Rimonabant were discontinued. Sibutramine is originally disclosed in U.S. Pat. No. 4,929,629 to Boots, incorporated by reference. Sibutramine is manufactured by Abbott Laboratories and is a neurotransmitter reuptake inhibitor. The use of Sibutramine for treating obesity is disclosed in U.S. Pat. No. 6,538,034 to Sepracor Inc., incorporated by reference. Orlistat is originally disclosed in U.S. Pat. No. 4,598,089 to Roche, incorporated by reference. Orlistat is manufactured by Roche and is a pancreatic lipase inhibitor. The use of Orlistat in treating obesity is also disclosed in U.S. Pat. No. 4,598,089.

Rimonanbant induces a (placebo-subtracted) net weight loss of about 6 kg (Despres et al., “Effects of Rimonanbant on metabolic risk factors in overweight patients with dyslipidemia”, N. Eng. J. Med, 353, 2121-2134, 2005, incorporated by reference), whereas Sibutramine and Orlistat produce a net weight loss of about 5 and 3 kg, respectively (Despres et al., “Effects of Rimonabant on metabolic risk factors in overweight patients with dyslipidemia”, N. Eng. J. Med. 353, 2121-2134, 2005; Li et al., “Pharmacologic treatment of obesity”, Ann. Intern. Med. 142, 532-546, 2005, both incorporated by reference,). Although weight reductions of this magnitude do produce some favourable metabolic effects, the improvements are modest and are insufficient in treating more obese mammals.

Combinations of the pharmaceutically active agents Rimonabant, Sibutramine or Orlistat with other active components are also known in the prior art. For example, U.S. Pat. No. 7,037,944 and U.S. Pat. No. 7,148,258, both to Sanofi-Aventis, incorporated by reference, teach the use of the combination of Sibutramine and a CB1 receptor antagonist for the treatment of obesity. US 2006/276549 to Abbott Laboratories, incorporated by reference, discloses the combination of sibutramine and rimonabant to treat obesity and obesity related disorders. Another example is US 2007/142369 to Schering Corp., incorporated by reference, which discloses the combination of a Histamine H3 antagonist and an appetite suppressant selected from the group consisting of a CB1 antagonist (e.g. rimonabant), sibutramine, phentermine and topiramate for the treatment of obesity and obesity related disorders. US 2005/124660 to Solvay, incorporated by reference, discloses the use of a combination of a pancreatic lipase inhibitor, e.g. Orlistat, and a CB1 receptor antagonist for the treatment of obesity. US 2006/269510 to Roche, incorporated by reference, discloses the combination of a lipase inhibitor, preferably Orlistat, and a bile acid sequestrant, for treating obesity. US 2006/135471 to Roche, incorporated by reference, discloses the combination of a lipase inhibitor and a glucomannan for the treatment of obesity. US2007/060532 to Fournier Laboratories Ireland Ltd., incorporated by reference, discloses the use of Orlistat and Metformin (an anti-diabetic drug) to treat patients suffering from obesity. US 2007/078179, also to Fournier Laboratories Ireland Ltd., incorporated by reference, discloses the use of a fibrate and Orlistat to treat patients suffering from obesity. However, the combinations do not seem to have been investigated in great detail yet and the existing evidence does not suggest significantly greater weight reduction compared to single-drug treatment (cf. Padwal, R. S. and Majumdar, S. R., The Lancet 369, 71-77, 2007, incorporated by reference).

Octreotide of Novartis (Sandostatin®) is originally disclosed in U.S. Pat. No. 4,395,403, incorporated by reference. Octreotide is an octapeptide that mimics natural somatostatin. Lustig, R. H. et al., Int. J. Obes. 30, 331-341, 2006 (“A multicenter, randomized, double blind, placebo-controlled, dose-finding trial of a long-acting formulation of octreotide in promoting weight loss in obese adults with insulin hypersecretion”), incorporated by reference, discloses that patients receiving 40 or 60 mg of octreotide LAR experienced statistically significant reduction in BMI of 0.73 and 0.79 kg/m², respectively.

Diaxozide (Proglycem®), a chemical compound of the group of 1,2,4-benzothiadiazine-1,1-dioxide derivatives, is a potassium channel activator and is used in the treatment of hypertension. The synthesis and its application as an anti-hypertensive pharmaceutical agent is disclosed in U.S. Pat. No. 2,968,573 and U.S. Pat. No. 3,345,365, both to Schering Corp., both incorporated by reference. Diaxozide is also known as a pharmaceutical agent for the treatment of secretory diarrhea (cf. U.S. Pat. No. 5,234,922 to the University of Iowa, incorporated by reference).

U.S. Pat. No. 5,234,922 discloses administering oral Diazoxide to an individual before ingestion of a food source in an amount effective to normalize the blood glucose and insulin levels. Diazoxide is administered in an amount from about 0.4 to about 0.8 mg/kg body weight before each meal. U.S. Pat. No. 5,234,922 discloses that low doses of Diazoxide, i.e. about 0.4 to about 0.8 mg/kg body weight, taken before each meal are effective to normalize blood glucose and insulin levels in subjects with a disturbance in the regulation of glucose and insulin levels that is characterised by the occurrence of postprandial hyperglycemia and reactive hypoglycemia. Paulsen does not provide any evidence that Diazoxide in this dose range is effective in the treatment of obesity or in suppressing the (fasting or peak) plasma insulin level.

Alemzadeh et al., “Beneficial Effect of Diazoxide in Obese Hyperunsulinemic Adults”, J. Clin. Endocrin. Metab. 83, 1911-1915, 1998, incorporated by reference, discloses the potential use of Diazoxide as a pharmaceutical agent to induce weight loss in humans. In a randomised, placebo-controlled trial with 24 hyperinsulinaemic patients (predominantly women), it was found that a relatively low dose of Diazoxide, i.e. maximal 200 mg/day, divided in three dosages) for eight weeks resulted in a significant higher weight loss than placebo (9.8 vs. 5.0 kg).

Schreuder et al., “Diazoxide-mediated insulin suppression in obese men: a dose-response study”, Diab. Obes. Metab. 7, 239-245, 2005, incorporated by reference, disclose the short-term Diazoxide-mediated insulin suppression in men in a one week dose-response study in order to determine the optimal dose in the treatment of obesity. Oral dosages at 150 and 225 mg per day (divided in three dosages) had no significant effect on plasma glucose and insulin levels, whereas an oral dosage of 300 mg per day (divided in three dosages), however, reduced plasma insulin levels by about 20% without affecting fasting or postprandial glucose levels. Consequently, it was found that insulin suppression is dose dependent, although the effect was found to be less in obese men than in non-obese men. Schreuder et al. suggested that effective insulin suppression in obese men would at least require a daily dose of 3.2 to 4.2 mg/kg body weight, i.e. for men in the weight range of 100-125 kg about 300 to 500 mg/day.

However, Due et al., “No effect of inhibition of insulin secretion by diazoxide on weight loss in hyperinsulinaemic obese subjects during an 8-week weight-loss diet”, Diab. Obes. Metab. 9, 566-574, 2007, incorporated by reference, could not confirm the results of Alemdazeh et al. in a study of similar design and duration, and also mainly including women (32 women, 3 men). Due et al. concluded that hyperinsulinaemia does not contribute to the maintenance of the obese state and that insulin secretion inhibition is presumably not a target for treating or preventing obesity. Due et al. also concluded from the study of Schreuder et al. that a daily dose of 3.2 to 4.2 mg/kg body weight would lead to worsen the hyperglycaemic state and would produce more undesired side-effects and would therefore not to be recommended in the treatment of obesity.

Van Boekel et al., Abstract book 67^{th} Annual Scientific Session American Diabetes Association June 2007, Chicago—Abstract 2742-PO, incorporated by reference, discloses an open, uncontrolled study to weight-loss by Diazoxide-induced insulin suppression in obese men (fourteen patients; four patients were eventually excluded). The dose of Diazoxide was started at 150 mg/day (divided in three equal dosages) and was raised every month to ultimately 900 mg/day (divided in three equal dosages), or until side effects occurred. After six months, the mean dosage was about 585 mg/day (plasma level of about 49 mg/l). Higher dosages were generally not tolerated due to side effects, in particular hyperglycaemia and edema. A body weight reduction was observed from about 114 kg to about 103 kg. However, the absolute suppressive effect on plasma insulin level is not disclosed.

U.S. Pat. No. 6,197,765 to Vardi and Morad, incorporated by reference, disclose the application of Diazoxide in the treatment of syndrome-X and resulting complications including hyperlipidemia, hypertension, central obesity, hyperinsulinaemia and impaired glucose intolerance. It is disclosed that Diazoxide mediated suppression of pancreatic insulin secretion is an effective treatment for “the metabolic syndrome” and for the prevention and treatment of diabetic complications in adult-onset diabetes mellitus. Hence, the invention disclosed in U.S. Pat. No. 6,197,765 is a combination treatment consisting of Diazoxide administered in increasing doses until endogenous insulinopenia appears which is then combined with administration of exogenous insulin.

WO 2006/026469 to Essentialis Inc., incorporated by reference, discloses oral, controlled release pharmaceutical formulations preferably comprising Diazoxide which may be used in the treatment of various disorders including diabetes and obesity. Paragraph [00382] suggests a daily dose of 100, 200 and 300 mg/day (divided in two dosages). Results are not disclosed.

WO 2006/045799 to Solvay, incorporated by reference, discloses pharmaceutical compositions comprising a CB1 antagonist and a potassium channel activator, e.g. Diazoxide. On page 29 it is disclosed that (4S)-3-(4-chlorophenyl)-N′-[(4-chlorophenyl) sulfonyl]-N-methyl-4-phenyl-4,5-dihydro-1-H-pyrazole-1-carboximidamide (indicated as Compound A) is more effective than Diazoxide at comparable dosages in inhibiting insulin secretion.

However, Van Boekel et al., Diabetes, Obesity and Metabolism 10(12), 1195-1203, 2008, demonstrated in a open, uncontrolled, 6-month pilot study that high-dose Diazoxide-mediated insulin suppression in combination with moderate caloric restriction and lifestyle advice resulted in a relevant degree of weight reduction in obese hyperinsulinaemic men. In particular, this study suggested that for effective weight loss the fasting insulin levels should be brought below 4.5 mU/l. Van Boekel et al. also suggested that this may be achievable with a combination of Diazoxide and Metformin.

Metformin (CAS No. 657-24-9) is known as a medicament for the treatment of diabetes (cf. U.S. Pat. No. 3,174,901, incorporated by reference,). Metformin has also been used for the treatment of obesity. For example, Kay et al., Metabolism 50(12), 1457-1461, 2001, conducted a double-blind placebo controlled clinical trial involving 24 hyperinsulinaemic, non-diabetic adolescents and showed that the metformin group showed about 6.5% weight loss compared to 3.8% in the placebo group at a dosage of 850 mg Metformin twice daily for eight weeks. Tankanova et al., Rom. J. Intern. Med., 41, 269-275, 2003, however, showed in a study involving 26 adults that at a dose of 2380±320 mg for six months) the weight reduction was only 3.24%. Consequently, the dosage effects and the efficacy of Metformin on weight reduction in obese subjects is not well understood.

Furthermore, US 2006/0240095, incorporated by reference, discloses a pharmaceutical composition comprising Metformin and a statin for the treatment of hyperglycemia non-insulin-dependent diabetes, dyslipidemia, hyperlipidemia, hypercholesterolemia and obesity.

WO 2008037807, incorporated by reference, discloses a pharmaceutical composition comprising Metformin and repaglinide for the treatment of non-insulin dependent diabetes mellitus (NIDDM) and the improvement of glycemic control.

There is still a need in the art for a more efficient and efficacious method for treating obesity.

It was surprisingly found that potassium channel activator-mediated, in particular Diazoxide-mediated plasma insulin suppression involving relatively low daily oral dosages of Diazoxide, in combination with daily oral dosages of an anti-diabetic drug, in particular Metformin, is very effective in reducing the plasma insulin levels without causing side-effects such as hyperglycemia or edema. This is an important finding because it indicates that the low plasma insulin levels required for clinically relevant weight loss can be achieved with lower doses of Diazoxide than required to obtain the same results with Diazoxide monotherapy. Thus, the combination of Diazoxide and Metformin may potentially reduce side effects associated with high dose Diazoxide monotherapy, and may be at least as effective or may be more effective as Diazoxide monotherapy in reducing plasma insulin levels.

### SUMMARY OF THE INVENTION

The present invention relates to the use of a combination of a potassium channel activator and an anti-diabetic drug in the manufacture of a medicament for suppressing the fasting and/or postabsorptive plasma insulin level in a mammal in need thereof, wherein the fasting and/or postabsorptive plasma insulin level is reduced to about 5 mU/l or less.

The present invention also relates to a pharmaceutical composition for suppressing the fasting and/or postabsorptive plasma insulin level in a mammal in need thereof, wherein the pharmaceutical composition comprises a combination of a potassium channel activator and an anti-diabetic drug, and wherein the fasting and/or postabsorptive plasma insulin level is reduced to about 5 mU/l or less.

The present invention also relates to a method for suppressing the fasting plasma insulin level and/or postabsorptive insulin level in a mammal in need thereof, said method comprising administering to said mammal a pharmaceutically effective amount of a combination of a potassium channel activator and an anti-diabetic drug.

The present invention further relates to a method for the prevention or treatment of obesity or obesity related disorders in a mammal in need thereof, said method comprising administering a combination of a potassium channel activator and an anti-diabetic drug.

The present invention also relates to a pharmaceutical composition for the prevention or treatment of obesity or obesity related disorders in a mammal in need thereof, said pharmaceutical composition comprising a combination of a potassium channel activator and an anti-diabetic drug.

### Definitions

The verb “to comprise” as is used in this description and in the claims and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there is one and only one of the elements. The indefinite article “a” or “an” thus usually means “at least one”.

All dosages of the potassium channel activator are given relative to a Diazoxide active weight basis, that is that every dosage of a potassium channel activator, e.g. in mg/kg or mg/kg/day, should be interpreted as whether the potassium channel activator was actually Diazoxide; the molecular weight of Diazoxide (7-chloro-3-methyl-4H-1,2,4-benzothiadiazine 1,1-dioxide) is 230.7 g/mol.

All dosages of the anti-diabetic drug are given relative to a Metformin active weight basis, that is that every dosage of a potassium channel activator, e.g. in mg/kg or mg/kg/day, should be interpreted as whether the anti-diabetes drug was actually Metformin. Metformin and its use as an anti-diabetic drug is disclosed in U.S. Pat. No. 3,174,901, incorporated by reference.

In this document, the term “postabsorptive” means the period between meals that starts after the ingested foods have been absorbed from the small intestine and there is no longer uptake of intestinal food components into the bloodstream.

### DETAILED DESCRIPTION OF THE INVENTION

### The Potassium Channel Activator

According to the present invention, the potassium channel activator is preferably a non-selective potassium channel activator, which is preferably selected from the group consisting of a 1,2,4-benzothiadiazine-1,1-dioxide derivatives. Such derivatives include not only the neutral organic compounds, but also their pharmaceutically acceptable addition salts, hydrates, solvates and polymorphs.

The 1,2,4-benzothiadiazine-1,1-dioxide derivatives can be represented by the general formula (I):

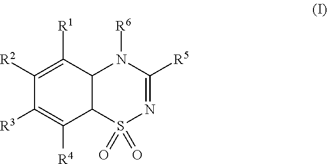

and tautomers thereof, wherein:
R¹, R², R³ and R⁴ are independently selected from the group consisting of hydrogen, halogen, C₁-C₆ alkyl and halogenated C₁-C₆ alkyl;
R⁵ and R⁶ are independently selected from the group consisting of hydrogen, C₁-C₆ alkyl, C₁-C₆ alkenyl and C₃-C₁₀ cycloalkyl.

Preferably, the 1,2,4-benzothiadiazine-1,1-dioxide derivatives are selected from the group consisting of the derivatives according to general formula (I), wherein R³ is a halogen, more preferably chlorine.

Its is also preferred that the 1,2,4-benzothiadiazine-1,1-dioxide derivatives are selected from the group consisting of the derivatives according to general formula (I), wherein R⁵ is a C₁-C₆ alkyl group, more preferably methyl.

Even more preferably, the 1,2,4-benzothiadiazine-1,1-dioxide derivatives are selected from the group consisting of:
7-chloro-3-methyl-4H-1,2,4-benzothiadiazine-1,1-dioxide (Diazoxide; CAS No. 364-98-7);
7-chloro-3-ethyl-4H-1,2,4-benzothiadiazine-1,1-dioxide;
6-chloro-3-methyl-4H-1,2,4-benzothiadiazine-1,1-dioxide;
3-n-propyl-6-trifluoromethyl-4H-1,2,4-benzothiadiazine-1,1-dioxide;
3-ethyl-6-trifluoromethyl-4H-1,2,4-benzothiadiazine-1,1-dioxide;
3-methyl-6-trifluoromethyl-4H-1,2,4-benzothiadiazine-1,1-dioxide;
7-bromo-3-isopropyl-4H-1,2,4-benzothiadiazine-1,1-dioxide;
6,7-dichloro-3-methyl-4H-1,2,4-benzothiadiazine-1,1-dioxide;
3-cyclopropyl-6,7-dichloro-4H-1,2,4-benzothiadiazine-1,1-dioxide;
7-chloro-3,6-dimethyl-4H-1,2,4-benzothiadiazine-1,1-dioxide;
6-chloro-3-n-butyl-4H-1,2,4-benzothiadiazine-1,1-dioxide;
6-chloro-3-n-pentyl-4H-1,2,4-benzothiadiazine-1,1-dioxide;
3,7-dimethyl-6-chloro-4H-1,2,4-benzothiadiazine-1,1-dioxide;
3-(3-cyclopentenyl)-6,7-dichloro-4H-4H-1,2,4-benzothiadiazine-1,1-dioxide;
6-chloro-3-(3-cyclopentenyl)-4H-1,2,4-benzothiadiazine-1,1-dioxide;
3-(3-cyclopentenyl)-6-trifluoromethyl-4H-1,2,4-benzothiadiazine-1,1-dioxide;
3-isopropylamino-4H-1,2,4-benzothiadiazine-1,1-dioxide;
3-isobutylamino-4H-1,2,4-benzothiadiazine-1,1-dioxide:
(2-ethylhexylamino)-4H-1,2,4-benzothiadiazine-1,1-dioxide;
cyclopropylamino-4H-1,2,4-benzothiadiazine-1,1-dioxide;
cyclohexylamino-4H-1,2,4-benzothiadiazine-1,1-dioxide;
7-chloro-3-(1,2,2-trimethylpropylamino)-4H-1,2,4-benzothiadiazine-1,1-dioxide;
7-chloro-3-(1,2-dimethylpropylamino)-4H-1,2,4-benzothiadiazine-1,1-dioxide:
7-chloro-3-(1-methylpropylamino)-4H-1,2,4-benzothiadiazine-1,1-dioxide;
7-chloro-3-isopropylamino-4H-1,2,4-benzothiadiazine-1,1-dioxide;
7-chloro-3-cyclopropylamino-4H-1,2,4-benzothiadiazine-1,1-dioxide;
7-chloro-3-cyclohexylamino-4H-1,2,4-benzothiadiazine-1,1-dioxide;
6-chloro-3-isopropylamino-4H-1,2,4-benzothiadiazine-1,1-dioxide;
6-chloro-3-cyclopropylamino-4H-1,2,4-benzothiadiazine-1,1-dioxide;
6,7-dichloro-3-isopropylamino-4H-1,2,4-benzothiadiazine-1,1-dioxide;
6,7-dichloro-3-cyclopropylamino-4H-1,2,4-benzothiadiazine-1,1-dioxide;
3-isobutylamino-7-methyl-4H-1,2,4-benzothiadiazine-1,1-dioxide;
3-cyclopentylamino-7-methyl-4H-1,2,4-benzothiadiazine-1,1-dioxide;
3-cyclohexylamino-6-trifluoromethyl-4H-1,2,4-benzothiadiazine-1,1-dioxide;
(N-cyclohexyl-N-methylamino)-4H-1,2,4-benzothiadiazine-1,1-dioxide;
3-cyclohexylamino-4-methyl-4H-1,2,4-benzothiadiazine-1,1-dioxide;
3-cyclohexylamino-2-methyl-2H-1,2,4-benzothiadiazine 1,1-dioxide;

Suitable 1,2,4-benzothiadiazine-1,1-dioxide derivatives are for example disclosed in U.S. Pat. No. 2,986,573, U.S. Pat. No. 3,345,365, U.S. Pat. No. 3,449,337, U.S. Pat. No. 3,462,428, U.S. Pat. No. 4,184,039 and U.S. Pat. No. 6,242,443, all incorporated by reference herein.

Most preferably, the potassium channel activator is Diazoxide.

### The Anti-Diabetic Drug

Many anti-diabetic drugs are known in the art. For example, several classes of anti-diabetic drugs are distinguished and the selection of a particular drug may depend on the nature of the diabetes, age and situation of the patient. Examples of such drugs include sulfonylureas, meglitinides, biguanides, thiazolidinediones, α-glucosidase inhibitors and peptide analogues (e.g. incretin mimetics, glucagon-like peptide analogs and agonists, gastric inhibitory peptide analogs, DPP-4 inhibitors and amylin analogues). Other examples of anti-diabetic drugs are PPARα/γ ligands, sodium-dependent glucose transporter 1 inhibitors and fructose 1,6-bisphosphatase inhibitors. According to the present invention, it is preferred that the anti-diabetic drug is a biguanide and pharmaceutically acceptable addition salts, hydrates, solvates and polymorphs thereof. The biguanide is in particular Metformin.

### Therapeutic Applications

As already disclosed above, it was found that the potassium channel activator-mediated plasma insulin suppression involving relatively high daily oral dosages of the potassium channel activator is very effective in reducing body weight in a mammal to a clinically relevant degree of weight reduction when the plasma insulin concentrations were lowered to particular levels. If such levels were not reached, the effect of the potassium channel activator-mediated plasma insulin suppression on body weight reduction is far less.

However, it was surprisingly found that the combination of a potassium channel activator and an anti-diabetic drug was at least as effective or more effective in reducing the plasma insulin levels and prevented the hyperglycaemic side effects of potassium channel activator monotherapy. Thus, the combination of the potassium channel activator and the anti-diabetic drug enables a lower dose of the potassium channel activator to achieve the desired plasma insulin level of 5 mU/l or less, than is required with potassium channel activator monotherapy.

Accordingly, a combination of the potassium channel activator and the anti-diabetic drug is administered to a mammal in need thereof for suppressing the fasting plasma insulin level in said mammal, wherein the fasting and/or postabsorptive plasma insulin level is reduced to about 5 mU/l or less. Similarly, a combination of the potassium channel activator and the anti-diabetic drug is administered to a mammal in need thereof for the prevention or treatment of obesity or obesity related disorders.

According to a first preferred embodiment of the present invention, the combination of the potassium channel activator and the anti-diabetic drug is administered to a mammal in need thereof to reduce or prevent weight gain. Consequently, the combination of the potassium channel activator and the anti-diabetic drug may be administered therapeutically or prophylactically. Hence, the present invention also relates to a method for preventing or treating obesity in a mammal in need thereof, said method comprising administering a combination of a potassium channel activator and an anti-diabetic drug to said mammal. Preferably, the method according to this first preferred embodiment of the invention includes the reduction of the fasting and/or postabsorptive plasma insulin level to about 5 mU/l or less.

According to a second preferred embodiment of the present invention, the combination of the potassium channel activator and the anti-diabetic drug is administered to a mammal in need thereof for the prevention or treatment of obesity, obesity related disorders and conditions and other disorders and conditions related to weight gain, wherein the combination of the potassium channel activator and the anti-diabetic drug may be administered therapeutically or prophylactically. Obesity, obesity related disorders and conditions and said other disorders and conditions related to weight gain are usually the result of an increase of fatty tissue in the mammal to a certain point at which it is associated with certain health conditions or even mortality. Such obesity related disorders and conditions and said other disorders and conditions include cardiovascular disorders, diabetes, in particular diabetes mellitus type 2, sleep apnea and osteoarthritis. Accordingly, the present invention also relates to a method of preventing or treating obesity, obesity related disorders and conditions and other disorders and conditions related to weight gain in a mammal in need thereof, said method comprising administering a combination of a potassium channel activator and an anti-diabetic drug to said mammal. Preferably, the method according to this second preferred embodiment of the invention includes the reduction of the fasting and/or postabsorptive plasma insulin level to about 5 mU/l or less.

The combination of the potassium channel activator and the anti-diabetic drug may be comprised by a single pharmaceutical composition which further comprises a pharmaceutically acceptable carrier or excipient. However, the potassium channel activator and the anti-diabetic drug may also be co-administered as two separate pharmaceutical compositions which further comprise a pharmaceutically acceptable carrier or excipient.

### Methods of Administration

Obviously and if desired, the administration may be intermittently (or cyclic) or may be continuous. Generally, cyclic regimens of administration are characterized as being intermittent, as opposed to continuous treatment regimens, and have both treatment periods during which a pharmaceutical composition is administered and non-treatment periods to permit the systemic level of active agent or active agents comprised by the pharmaceutical composition to return to baseline. For example, the administration may be continued for a period of six months followed by a period of two months where a pharmaceutical composition is not administered, where after the administration is started again for a period of six months. A continuous regimen of administration involves daily administration, twice-weekly administration, once-weekly administration, once every two weeks administration and the like.

According to the present invention, however, it is preferred that the combination of the potassium channel activator and the anti-diabetic drug is administered continuously, preferably daily, preferably in the form of an oral medicament to the mammal in need thereof.

It is also preferred according to the present invention that the combination of the potassium channel activator and the anti-diabetic drug is administered to the mammal in need thereof for prolonged periods. Preferably, the combination of the potassium channel activator and the anti-diabetic drug is administered for a period of one month to twenty four months, more preferably one month to twelve months.

According to a preferred embodiment of the present invention, the combination of the potassium channel activator and the anti-diabetic drug potassium channel activator is administered to the mammal in need thereof in such an mount that a serum level of the potassium channel activator is achieved of about 20 mg/l or more, calculated on a Diazoxide active weight basis, preferably 40 mg/l or more and even more preferably 45 mg/l or more. It is also preferred that the serum level of the potassium activator does not exceed 100 mg/l, calculated on a Diazoxide active weight basis, preferably 80 mg/l. Accordingly, the present invention also relates to a method for preventing or treating weight gain in a mammal in need thereof, said method comprising administering a combination of a potassium channel activator and an anti-diabetic drug to said mammal in an amount sufficient to achieve a serum level of the potassium channel activator in said mammal of about 20 mg/l or more, calculated on a Diazoxide active weight basis. Likewise, the present invention also relates to a method of preventing or treating obesity, obesity related disorders and conditions and other disorders and conditions related to weight gain in a mammal in need thereof, said method comprising administering a combination of a potassium channel activator and an anti-diabetic drug to said mammal in an amount sufficient to achieve a serum level of the potassium channel activator in said mammal of about 20 mg/l or more, calculated on a Diazoxide active weight basis. In both methods it is preferred that the serum level of the potassium activator does not exceed 100 mg/l, calculated on a Diazoxide active weight basis.

According to the present invention, the serum level of the potassium channel activator in said mammal of about 20 mg/l or more is maintained for at least one month, preferably for one month to twenty four months, even more preferably one month to twelve months.

Furthermore, the combination of the potassium channel activator and the anti-diabetic drug may be administered to a mammal in need thereof in relatively low dosages according to cyclic or continuous administration regimens to reduce weight gain. The combination of the potassium channel activator and the anti-diabetic drug may also be administered to a mammal in need thereof in relatively low dosages and according to cyclic or continuous administration regimens to prevent weight gain. Furthermore, the combination of the potassium channel activator and the anti-diabetic drug may be administered to a mammal in need thereof in relatively low dosages and according to cyclic or continuous administration regimens to reduce weight gain or in the treatment of a disorder or condition associated with weight gain. Such disorders include obesity, diabetes and the like.

### Administration Schemes and Dosages

According to the invention, it is preferred that the combination of the potassium channel activator and the anti-diabetic drug is administered to a mammal in need thereof over prolonged periods, preferably from one month to twenty four months, more preferably from one month to twelve months.

It is also preferred that the combination of the potassium channel activator and the anti-diabetic drug or the pharmaceutical composition comprising said combination is administered orally.

The dosages of the combination of the potassium channel activator and the anti-diabetic drug need to be sufficient to achieve a serum level of the potassium channel activator in the mammal of about 20 mg/l or more as otherwise a reduction of the fasting and/or postabsorptive plasma insulin level to about 5 mU/l or less, is not achieved. Obviously, this is also dependent from the disorder or condition to be treated or prevented.

Accordingly, the potassium channel activator is in general administered in a daily dosage of about 5 mg to about 1000 mg, calculated on a Diazoxide active weight basis, wherein the anti-diabetic drug is co-administered in a daily dosage of about 500 mg to about 5000 mg, calculated on a Metformin active weight basis. This implies that at a molar level the potassium channel activator is administered in a daily dosage of about 0.0217 mmol to about 4.335 mmol and that the anti-diabetic drug is administered in a daily dosage of about 3.871 mmol to about 38.71 mmol. However, it is preferred that the potassium channel activator is administered in a daily dosage of about 50 mg to about 900 mg, more preferably about 150 to about 900 mg, yet even more preferably about 300 mg to about 800 mg and most preferably about 400 mg to about 700 mg. These dosages are based on a BMI of a mammal of about 30 to about 35 kg/m².

The anti-diabetic drug is preferably co-administered in a daily dosage of about 750 mg to about 4000 mg, more preferably in a daily dosage of about 1000 mg to about 3000 mg, calculated on a Metformin active weight basis.

According to a preferred embodiment of the invention, the mammal is administered a daily dosage of about 15 mg to about 300 mg, preferably about 75 mg to about 225 mg, more preferably about 100 mg to about 225 mg, even more preferably about 125 mg to about 225 mg, of the potassium channel activator, calculated on a Diazoxide active weight basis, for the first two to six weeks, wherein the dosage level is increased every two to six weeks with about 15 mg to about 300 mg, preferably about 75 mg to about 225 mg, more preferably about 100 mg to about 225 mg, even more preferably about 125 mg to about 225 mg, of pharmaceutical composition. Preferably, the daily end-dosage does not exceed 1000 mg of the potassium channel activator. If desired, these dosages may be divided over two or three dosages during the day. This dosage regimen enables the physician to control the serum level of the potassium channel activator and the fasting and/or postabsorptive insulin level during the prescription of the combination of the potassium channel activator and the anti-diabetic drug according to the present invention, thereby enabling him to adjust the dosage level to an optimum for the mammal concerned. As it will be clear to the person skilled in the art, however, that the dosage regimen is also dependent from the BMI of the mammal. Hence, mammals having a relatively lower BMI, in particular below 30 kg/m², but higher than 25 kg/m² (i.e. “overweight”; cf. World Health Organization. Technical report series 894: “Obesity: preventing and managing the global epidemic.”, Geneva, World Health Organization, 2000) may presumably need a lower dosage than mammals having a relatively higher weight, i.e. 30 kg/m² or higher. Consequently, in terms of BMI, the daily dosage is preferably about 5 mg to about 900 mg when the BMI of the mammal is 25-30 kg/m², more preferably about 50 mg to about 800 mg, even more preferably about 100 to about 800 mg, and most preferably 150 to about 700 mg of the potassium channel activator. Obviously, such dosage regimen may also be intermittently (or cyclic) or may be continuous as is explained above. These dosage schemes may in particular be important for the prevention of disorders and conditions related to weight gain as is explained above. These dosages of the potassium channel activator are preferably combined with dosages of the anti-diabetic drug, wherein the weight ratio of the daily administered potassium channel activator:anti-diabetic drug is preferably in the range of about 1:about 2 to about 1:about 10.

According to a further embodiment of the present invention, the combination of the potassium channel activator and the anti-diabetic drug is administered to the mammal in need thereof is administered continuously or for as long as desired to maintain the established weight reduction obtained after a treatment as disclosed above. Obviously, according to this embodiment, the dosages of the potassium channel activator and/or of the anti-diabetic drug may be lower than in the administration regimens disclosed above.

### Pharmaceutical Compositions

Pharmaceutical compositions (or medicaments) comprising a potassium channel activator are known from the prior art. For example, Proglicem® is available as capsules containing 100 mg of Diazoxide. However, such pharmaceutical compositions comprise relatively low amounts of the potassium channel activator. For the medical uses indicated in this document, however, generally higher dosages are required. Likewise, oral formulations, e.g. tablets, of Metformin contain generally dosages of 500 mg, 850 mg, or 1000 mg. However, a higher efficacy of dosages up to 5000 mg per day, in particular in obese men, may be necessary to obtain the optimal weight reducing effect.

Although the potassium channel activator and the anti-diabetic drug can be administered as separate pharmaceutical compositions, it is preferred, e.g. for patient compliance, that the potassium channel activator and the anti-diabetic drug are administered as a single pharmaceutical composition.

Accordingly, this invention also relates to pharmaceutical compositions comprising a potassium channel activator, an anti-diabetic drug and a pharmaceutically acceptable carrier or excipient, wherein the pharmaceutical composition comprises about more than 100 mg, preferably 150 mg to about 1000 mg of the potassium channel activator, calculated on a Diazoxide active weight basis. It is, however, preferred that the pharmaceutical composition comprises about 150 mg to about 900 mg, more preferably about 200 to about 900 mg, yet even more preferably about 300 mg to about 800 mg and most preferably about 400 mg to about 700 mg of the potassium channel activator. These pharmaceutical compositions further comprise about 750 mg to about 4000 mg, more preferably about 1000 mg to about 3000 mg of the anti-diabetic drug, calculated on a Metformin active weight basis.

These pharmaceutical compositions are very suitable for daily administration. As it will be obvious to the person skilled in the art, such pharmaceutical compositions may also comprise only about one third of the active ingredients if the pharmaceutical composition is intended to be administered three times a day. Accordingly, the pharmaceutical compositions or medicaments may be administered once, two times or three times per day. Alternatively, if the pharmaceutical composition is intended to be administered in e.g. a once-weekly manner, the pharmaceutical composition may comprise the active ingredients in a relatively higher dosage.

### The Subject

According to the present invention, the mammal to be subjected to a treatment or a prevention of obesity, obesity related disorders and conditions and other disorders and conditions related to weight gain, is preferably human, more preferably male, and is most preferably a hyperinsulinemic obese man. It is also preferred that the fasting and/or postabsorptive plasma insulin level in said mammal is reduced to about 5 mU/l or less, wherein said mammal is subjected to such a (prophylactic or systemic) treatment.

### EXAMPLE

The first study was designed to test the concept that Diazoxide mediated insulin suppression is associated with substantial weight loss. Fourteen obese, healthy men were studied for 6 months in an open, uncontrolled study. They were 30-50 years of age, had a BMI of above 30 to about 35 kg/m², a stable body weight for at least three months, a HbAlc <6.0% and a fasting plasma C-peptide >1.0 nmol/l.

All subjects received dietary advice to reduce caloric intake to meet the calculated basal requirements for ideal body weight (based on the Harrison-Bennedict equation). They were also instructed to increase walking exercise to at least 30 minutes a day. After completion of the baseline measurements, the subjects started Diazoxide 50 mg (three times a day) which was taken before each meal. Subsequently, the following data were recorded every month, for a period of 6 months: body weight, abdominal circumference, supine and upright blood pressure, the results of a two-day, 8-point HGM, and adverse effects. At every monthly visit, the daily Diazoxide dose was increased by 3×50 mg until a maximum of 300 mg (three times a day) was reached or until adverse side effects occurred. The Diazoxide dose was not increased if clinically relevant edema persisted for more than a month, if upright systolic blood pressure was <110 mm Hg, if upright diastolic blood pressure was <70 mm Hg, or if fasting home glucose level was >7 nmol/l, or non-fasting home glucose level was >11 nmol/l.

### Results

The estimated caloric intake prior to the study was 2242±125 kcal/day and comprised 48% carbohydrates, 32% fat, and 20% proteins. The recommended intake during the study was 1499±48 kcal/day. This represents a reduction of 31.3±3.4% compared to pre-study intake (P<0.001). The mean fasting insulin level at baseline was 18.0±2.3 mU/l. This is about 3 times the normal value in age-matched, non-obese men. Mean HbAlc at baseline was 5.4±0.1%.

### DZX Dose and Side Effects

DZX was increased every month until a maximum of 900 mg/day was reached or until side effects occurred. At six months the mean daily dose had increased to 600±54 mg (5.7±0.5 mg/kg). Individually tolerated DZX doses ranged from 300 to 900 mg/day: 3 men tolerated only 300 mg/day, whereas only 2 men reached the 900 mg dose. Hyperglycaemia (n=4) and edema (n=8) were the main dose limiting events. Other adverse effects were not observed.

### Effects on Serum Glucose and Insulin Levels

DZX reduced fasting insulin levels by 65% (P<0.001). Fasting glucose increased by 0.8±0.3 mmol/L (P<0.01).

### Effects on Body Composition

Body weight gradually decreased from 115.1±3.4 to 105.7±3.8 kg (−9.4 kg, −8.3%, P<0.001). Waist circumference decreased from 116.9±1.4 to 107.7±2.2 cm (−9.2 cm, −7.9%, P<0.001). Total body fat mass decreased by 9.5±1.9 kg (−23.3%, P<0.001), without a concomitant change in soft tissue lean body mass or bone mass. The change in body weight was inversely correlated with fasting insulin levels at 6 months (R −0.59, P=0.023). The change in fat mass as measured by DEXA was also related to the fasting insulin levels at 6 months (R −0.76, P=0.002) As shown in FIG. 1, substantial loss of body fat, i.e. a decrease of 10 kg or more, only occurred if plasma insulin levels were below 4.5 mU/L, which is equivalent to about 30 pmol/L in this assay (conversion of mU/L to pmol/L:multiply by 6.945).

From this study it was concluded that the degree of weight loss is determined by the fasting insulin level that can be achieved during treatment, i.e. the lower the insulin level, the higher the amount of weight loss will be.

The second study was designed tot test the concept that the addition of Metformin 1700-2550 mg/day to Diazoxide has beneficial effects on blood glucose or insulin levels in Diazoxide treated subjects. The study was focused on detecting the early beneficial effects. Predefined benefits of the combination Diazoxide+Metformin versus Diazoxide monotherapy were: prevention of hyperglycemia, and/or a more profound reduction in serum insulin levels during treatment. Addition of Metformin was expected to induce a more profound decrease in serum insulin levels and thus cause a greater degree of weight loss in the long term (see FIG. 1). The study was double-blind, and placebo-controlled and had a duration of 2 months. There were 3 treatment groups: Diazoxide+Metformin (DZX+MTF: 5 men), Diazoxide+Placebo (DZX+PL: 7 men), and Placebo+Placebo (PL+PL: 7 men). Diazoxide was started in a dose of 100 mg twice a day, and was increased to 100 mg three times a day in the second month. Metformin was started in a dose of 850 mg twice daily, and was increased to 850 mg three times daily in the second week. All subjects received exercise training three times a week, and all were instructed to take a 30 minute walk after lunch and after dinner.

Dietary intake was reduced by about 30%, according to the calculation procedure described in example 1.

A total of 19 obese men with a BMI of 30-37.5 kg/m² participated in the study. PL+PL treatment did not affect serum glucose and insulin levels significantly (FIG. 2). DZX+PL treatment was associated with a decrease in fasting serum insulin of 42.4±17.7% (mean±standard error of the mean, SEM) and a rise in serum glucose level from 5.7±0.2 mmol/L to 7.3 to 1.2 mmol/L. In contrast, DZX+MTF induced a decrease in serum insulin of 57.2±5.3%, and was not associated with a rise in serum glucose levels.

FIG. 2. Comparison of serum insulin and glucose responses in obese men treated with placebo (PL+PL), Diazoxide monotherapy (DZX+PL) and the combination of Diazoxide and Metformin (DZX+MTF).

It was concluded that 1. DZX+MTF caused a greater suppression of serum insulin levels than Diazoxide monotherapy, and that 2. DZX+MTF had no adverse effects on serum glucose levels whereas Diazoxide monotherapy was associated with a substantial rise in serum glucose levels which would limit a further increase in Diazoxide dose in the DZX monotherapy group. The latter observation is likely to have implications for the longterm efficacy of treatment. A significant rise in serum glucose level is a dose limiting event during Diazoxide treatment, and this is likely to reduce the degree of insulin suppression that can be induced by Diazoxide monotherapy. The combination of Diazoxide and Metformin induces a greater reduction of serum insulin than can be achieved with Diazoxide monotherapy, and thus is expected to cause a greater reduction in body weight (see study 1, FIG. 1). Because of Metformin's synergistic effects on serum insulin levels, a lower dose of Diazoxide is required to achieve a suppression of plasma insulin below 5 mU/l ore less. Therefore, the addition of Metformin may serve to improve the efficacy weight loss treatment and reduce the Diazoxide related side effect of hyperglycemia and edema formation.

1-36. (canceled)

37. A method for suppressing the fasting plasma insulin level and/or postabsorptive insulin level in a mammal in need thereof, said method comprising administering to said mammal pharmaceutically effective amounts of a potassium channel activator and an anti-diabetic drug.

38. The method according to claim 37, wherein the anti-diabetic drug is a biguanide.

39. The method according to claim 38, wherein the biguanide is Metformin.

40. The method according to claim 37, wherein the fasting and/or postabsorptive plasma insulin is reduced to about 5 mU/l or less.

41. The method according to claim 37, wherein the potassium channel activator is a non-selective potassium channel activator.

42. The method according to claim 38, wherein the potassium channel activator is a 1,2,4-benzothiadiazine-1,1-dioxide derivative.

43. The method according to claim 42, wherein the potassium channel activator is Diazoxide.

44. The method according to claim 37, wherein the potassium channel activator medicament is orally administered.

45. The method according to claim 37, wherein the anti-diabetic drug is orally administered.

46. The method according to claim 37, wherein the potassium channel activator is administered to the mammal at a daily dosage of about 5 mg to about 1000 mg, calculated on a Diazoxide active weight basis.

47. The method according to claim 37, wherein the potassium channel activator is administered one, two or three times a day.

48. The method according to claim 37, wherein the anti-diabetic drug is administered to the mammal at a daily dosage of about 500 mg to about 5000 mg, calculated on a Metformin active weight basis.

49. The method according to claim 37, wherein the anti-diabetic drug is administered one, two or three times a day.

50. The method according to claim 37, wherein the mammal is human.

51. The method according to claim 50, wherein the human is male.

52. The method according to claim 51, wherein the human male is hyperinsulinaemic obese.

53. The method according to claim 37, wherein the peak plasma insulin level is to be reduced to about 50 mU/l or less.

54. The method according to claim 37, wherein the potassium channel activator and the anti-diabetic drug are co-administered for at least one month.

55. A method for treating or preventing obesity in a mammal in need thereof, said method comprising administering to said mammal pharmaceutically effective amounts of a potassium channel activator and an anti-diabetic drug.

56. A method for treating or preventing obesity, obesity related disorders and conditions and other disorders and conditions related to weight gain in a mammal in need thereof, said method comprising administering to said mammal pharmaceutically effective amounts of a potassium channel activator and an anti-diabetic drug.
